(19) United States
(12) Patent Application Publication
(10) Pub. No.: US 2023/0371810 A1
(43) Pub. Date: Nov. 23, 2023

(54) APPARATUS AND METHOD FOR ATTACHING A HANDS-FREE LENS TO A MICROSCOPE FOR USE DURING OCULAR SURGERY

(71) Applicant: OCULUS SURGICAL INC., Port St. Lucie, FL (US)

(72) Inventors: Michael ANNEN, Ft. Pierce, FL (US); Steffen ADAMOWICZ, Solms (DE)

(21) Appl. No.: 18/027,746

(22) PCT Filed: Sep. 22, 2021
(86) PCT No.: PCT/US2021/051508

§ 371 Date: Mar. 22, 2023

Related U.S. Application Data
(60) Provisional application No. 63/081,469, filed on Sep. 22, 2020.

(51) Int. Cl. A61B 3/117; G02B 21/00; A61B 3/13

(52) U.S. Cl. CPC A61B 3/117; G02B 21/0012; A61B 3/13

(57) ABSTRACT

An apparatus is presented for attaching a lens to a microscope with an optical attachment. The apparatus includes the lens with a translational degree of freedom such that the lens is configured to translate along a first direction relative to the microscope and the optical attachment. A system is also presented that includes the apparatus and the optical attachment. The optical attachment includes a lens holder and/or the viewing attachment configured to move the lens and the lens holder relative to the microscope. A method is also presented for using an optical attachment to position a hands-free lens relative to a microscope.

### CROSS-REFERENCE TO RELATED APPLICATIONS

This application claims benefit of U.S. Provisional Application No. 63/081,469, filed Sep. 22, 2020, under 35 U.S.C. § 119(e).

### BACKGROUND

While observing the eye of a patient under a microscope, medical professionals may place an additional optic in contact with the cornea to improve their view of an intraocular structure. Various optics are known which accommodate an ophthalmologist's view of an eye in different ways. For example, a gonioscopy lens provides an ophthalmologist with an angled view through the cornea that allows visualization of the peripheral sections of the anterior chamber that are otherwise difficult to visualize.

### SUMMARY

Primary Open Angle Glaucoma is a disease state characterized by elevated intraocular pressures, the cause of which is most commonly attributed to a restricted outflow pathway through the trabecular meshwork and Schlemm's canal. These anatomical structures are located within the iridocorneal angle 901 (see FIG. 9A) in the periphery of the anterior chamber. When surgical intervention is required to increase aqueous outflow, precise visualization of these fine structures is needed. The iridocorneal angle is normally not visible from the eye's exterior due to an optical phenomenon known as Total Internal Reflection (TIR). FIG. 9B is a ray diagram that illustrates an example of light refraction and total internal reflection at a boundary between materials 903, 905 of different index of refraction n₁, n₂ where n₁ is greater than n₂. In one embodiment, the first material 903 is the eye (e.g. cornea) or a solution along the eye surface (e.g. tear) and the second material 905 is air. As shown in the left side of FIG. 9B, an incident ray from within the eye is incident at the eye-air interface at a first angle φ₁ (e.g. measured relative to a normal 907 to the interface) and is refracted as a refracted ray at a second angle φ₂ (e.g. also measured relative to the normal 907) that is greater than the first angle φ₁. As shown in the middle of FIG. 9B, an incident ray from within the eye is incident at the eye-air interface at a first angle φ₁ (e.g. greater than the first angle φ₁ in the left side of FIG. 9B) and is refracted along the boundary of the eye-air interface (e.g. the second angle φ₂ is 90 degrees). The first angle φ₁ in the center of FIG. 9B is known as the “critical angle” since incident light from within the eye on the eye-air interface is refracted along the eye-air interface. As shown in the right side of FIG. 9B, an incident ray from within the eye is incident on the eye-air interface at a first angle φ₁ that is greater than the critical angle and the incident ray is reflected (TIR) at the eye-air interface back into the eye at a second angle φ₂ that is equal to the first angle φ₁. As shown in the right side of FIG. 9B, TIR occurs at a boundary between the two materials 903, 905 with a difference in index of refraction, when the incident angle φ₁ exceeds the critical angle (center of FIG. 9B). If the ray approaches such a boundary at a shallow enough angle φ₁, (e.g. equal to or greater than the critical angle), it is possible that the ray exiting into the second material 905 (e.g. air) with the lower index of refraction n₂ would be refracted such that the angle of refraction theoretically would be greater than 90 degrees (e.g. when the incident angle φ₁ is the critical angle) or beyond parallel to the boundary thus becoming a reflection rather than refraction (e.g. when the incident angle φ₁ exceeds the critical angle). The critical angle for the tear-air interface is about 46°. If light from the interior of the eye strikes the cornea at an angle shallower than 46° (e.g. if the incident angle φ₁ is greater than the critical angle for the eye-air interface), TIR will occur and light will not exit the eye.

As shown in FIG. 10, a hand-held gonioscopy lens 1000 in principle acts as a continuation of the cornea and permits light from the iridocorneal angle to cross the air boundary at an angle closer to perpendicular. The gonioscopy lens 1000 includes a first surface 1003 (e.g. in contact with the eye 115) and a second surface 1005 (e.g. in contact with the air). Since the lens 1000 acts as a continuation of the cornea, and thus there is minimal difference in the index of refraction between the lens 1000 and the cornea, no TIR occurs at the lens-cornea boundary. Additionally, since the normal to the second surface 1005 is substantially aligned with the incident light within the lens 1000 to the lens-air boundary, the incident angle of the incident light on the lens-air boundary is relatively small in magnitude and thus no TIR occurs at the lens-air boundary. To avoid pockets of air between the first surface 1003 and the eye 115, generally an ophthalmic solution is applied onto the first surface 1003 prior to placing the gonioscopy lens 1000 in contact with the eye 115. The surgeon (or an assistant) holds the lens 1000 on the eye 115 This permits the surgeon to view the interior anatomical structures of the eye at the iridocorneal angle 901.

The inventors of the present invention recognized that the majority of optics used for optical procedures (e.g. a majority of gonioscopy lenses used for anterior surgical procedures) are hand-held lenses that must be manually retained in position on the cornea. In most cases, the surgeon operates with the handheld gonioscopy lens in one hand and a surgical instrument in the other. In straightforward procedures (e.g. bypass shunt placement) this is an effective way to perform the surgery as the surgeon has direct control of the view and the instrument simultaneously. In more complex procedures, limiting the surgeon to use of one hand increases the time and difficulty of the procedure. For this reason, in some cases it may be beneficial or even necessary for the surgeon to be able to operate bimanually using a second instrument. In order to do so, the handheld gonioscopy lens is generally held by an assistant with the understanding that the lens will frequently need to be repositioned through verbal instructions.

The inventors of the present invention recognized that some lenses offer self-stabilization features, e.g. a flange along the lower lens surface that extends to increase the base of the lens. The inventors recognized that while the stabilization features will improve the lens retention, adjustments of the lens will likely still be needed, requiring the surgeon to remove an instrument in order to manually reposition the lens. The inventors also recognized that a flange also presents a different issue in that it can restrict access to various insertion points. The flange may also impede visualization. The inventors of the present device realized the need for an alternative self-stabilizing lens that could be used without the aid of an assistant and enable true bi-manual surgery.

One instance of a microscope suspended gonioscopy lens has been identified in U.S. Patent Publication Number 2013/0182223. The design of this complex system centers on a counterweight style lens holder. However, the inventors of the present invention noticed drawbacks of this suspended lens design including that the lens only has one rotational degree of freedom (about one rotational axis) relative to a lens holder and attachment that is used to suspend the lens from the microscope and that the lens does not feature a translational degree of freedom relative to the attachment. Thus, the inventors of the present invention developed an improved lens holder design herein which features multiple rotational degrees of freedom (about two or more rotational axes) and a translational degree of freedom of the lens relative to the lens holder and an attachment that suspends the lens to the microscope.

Another instance of a suspended gonioscopy lens has been identified in U.S. Pat. No. 8,118,431 ('431 patent hereafter). This design however specifies the attachment to the objective lens of the microscope in its description and abstract. It also focuses on using a mirrored gonioscopy lens and attachment configured to position the lens between the microscope and the eye to simultaneously view the surface and the interior of the eye (a claim taught in U.S. Pat. No. 4,157,859 to Terry, as well as in US20060098274 Kitajima). The '431 patent fails to teach how the lens is suspended from the objective lens to provide a method for compensation of patient eye movement, misalignment of the eye relative to the microscope optical axis, and the necessary safety feature to prevent patient trauma in the event of unintended large microscope movement. Thus, the inventors of the present invention developed the improved lens holder and lens design herein, to overcome these noted drawbacks in the '431 patent.

In vitreo-retinal procedures, or procedures in the posterior chamber of the eye, the inventors recognized that a wide-angle viewing attachment (“viewing attachment” herein) is often used on the ophthalmic operating microscope. A wide-angle viewing attachment is typically mounted to the body of the microscope and suspends a wide-angle lens below the microscope objective, in close proximity to the corneal surface. Though the viewing attachment is not intended to hold the lens in contact with the cornea, the inventors of the present device realized that this could be an effective method of positioning and retaining a lens that would contact the cornea.

The assignee of the present invention (OCULUS GmbH) manufactures wide-angle viewing attachments and adapters to mount to a variety of operating microscopes. One embodiment of the present invention employs a wide-angle viewing attachment and adapter in a method for attaching a novel apparatus (e.g. for positioning a hands-free lens on the cornea) to various operating microscopes.

One type of wide angle-viewing attachment requires sterilization in between uses. In an example, the assignee developed an example of this wide-angle viewing attachment (OCULUS® Binocular Indirect Ophthalmomicroscope or “OCULUS BIOM” herein, and disclosed in U.S. Pat. No. 7,092,152 which is incorporated by reference herein).

Another type of wide angle-viewing attachment is for use as a single-use disposable. In an example, the assignee developed an example of this wide-angle viewing attachment (OCULUS Binocular Indirect Ophthalmomicroscope Ready or “BIOM READY” herein and disclosed in U.S. Pat. No. 9,155,593 which is incorporated by reference herein). In one example, the BIOM READY wide-angle viewing attachment is injection molded and is for use as a single-use disposable.

In one embodiment, the inventors recognized that it would be advantageous to provide an apparatus that attaches a hands-free lens to a wide angle-viewing attachment, such that the apparatus permits the lens to contact the eye without the need to manually hold the lens. The inventors recognized that it would be further advantageous if such an apparatus is designed to accommodate relative movement between the eye and the wide-angle viewing attachment (and/or microscope) along multiple degrees of freedom (e.g. translational and/or rotational). In an example embodiment, the apparatus is made for use with any viewing attachment, such as the OCULUS BIOM or the BIOM READY wide angle viewing attachments. With the BIOM READY wide angle viewing attachment, the apparatus can be used as an all-encompassing disposable system.

Advantageous embodiments of the proposed invention disclose a means to attach a lens (e.g. surgical contact lens) to a wide-angle viewing attachment (e.g. OCULUS BIOM, BIOM READY, etc.) in a method that allows for stable and hands-free positioning of the lens atop the cornea.

In a first set of embodiments, an apparatus is presented for attaching a lens to a microscope with an optical attachment. The apparatus includes the lens with a translational degree of freedom such that the lens is configured to translate along a first direction relative to the microscope and the optical attachment.

In a second set of embodiments, a system is presented for attaching a lens to a microscope with an optical attachment. The system includes a lens and the optical attachment to attach the lens to the microscope. The optical attachment includes a lens holder and/or the viewing attachment configured to move the lens and the lens holder relative to the microscope.

In a third set of embodiments, a method is presented for using an optical attachment to position a lens relative to a microscope. The method includes securing the lens to a first end of the optical attachment and securing a second end of the optical attachment to the microscope. The method also includes moving the lens with the optical attachment until the lens makes contact with an eye of a patient. The method also includes translating the lens along a first direction relative to the microscope and the optical attachment, based on relative movement of the eye in the first direction such that the lens maintains contact with the eye.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIG. 1 is an image that illustrates an example of a system providing a hands-free lens for use during optical surgery, according to an embodiment;

FIG. 2A is an image that illustrates an example of a side perspective view of the lens and lens holder of the system of FIG. 1, according to an embodiment;

FIG. 2B is an image that illustrates an example of a side perspective view of the lens and lens holder of the system of FIG. 1, according to an embodiment;

FIG. 3 is an image that illustrates an example of a side perspective view of the lens of FIG. 2A or 2B, according to an embodiment;

FIG. 4 is an image that illustrates an example of a top perspective view of the lens holder of FIG. 2A or 2B, according to an embodiment;

FIG. 5 is an image that illustrates an example of a side detail view of the connection of the lens holder to the viewing attachment of FIG. 1, according to an embodiment;

FIG. 6 is an image that illustrates an example of a system providing a hands-free lens for use during optical surgery, according to an embodiment;

FIGS. 7A through 7C are images that illustrate an example of the viewing attachment of FIG. 1 being rotated about the optical axis of the microscope, according to an embodiment;

FIG. 8A is an image that illustrates an example of the lens of FIG. 1 configured to rotate about a first rotational axis relative to the microscope, according to an embodiment;

FIG. 8B is an image that illustrates a cross-sectional view of FIG. 8A, showing the lens configured to rotate about a second rotational axis relative to the microscope, according to an embodiment;

FIG. 9A is an image that illustrates an example of interior anatomy of the human eye;

FIG. 9B is a ray diagram that illustrates an example of light refraction and total internal refraction at a boundary between materials of different indices;

FIG. 10 is an image that illustrates an example of a gonioscopy lens manually held on the eye of a subject;

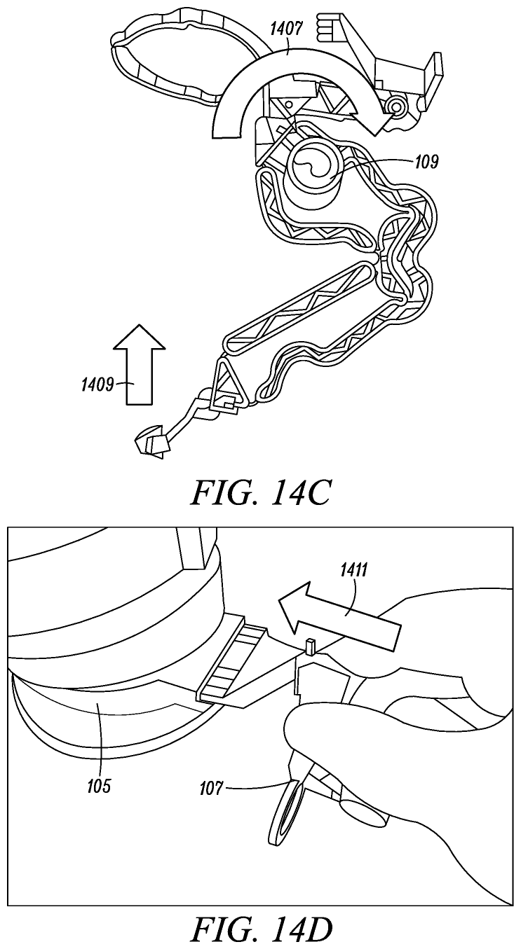

FIGS. 11 and 12 are images that illustrate an example of different views of the system of FIG. 1 and FIG. 6, according to embodiments;

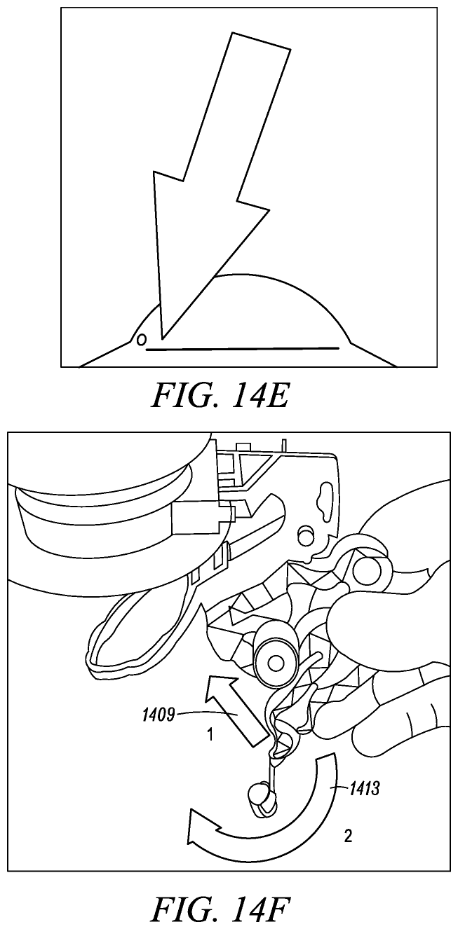

FIG. 13 is a flowchart that illustrates an example of a method for providing a hands-free lens for use during optical surgery, according to an embodiment; and

FIGS. 14A through 14J are images that illustrate an example of performing one or more of the steps of the method of FIG. 13, according to an embodiment;

### DETAILED DESCRIPTION

A method and apparatus are described for attaching a lens to a microscope with an optical attachment (e.g. for use during a surgical procedure). In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about X” implies a value in the range from 0.5 X to 2 X, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

Some embodiments of the invention are described below in the context of optical devices used to treat or examine a patient (e.g. examine the patient, perform surgery on the patient, etc.). In some embodiments, the invention is described in a context of a system provided including a lens, and an apparatus to position the lens and secure the lens to the body of an ophthalmic operating microscope. In one embodiment, the system is intended to safely position the lens onto the eye in a way that is stable and non-obstructive for the user, avoiding the need to manually hold a lens. In another embodiment, a method is provided for using the microscope with the addition of the system, including its installation. In yet another embodiment, a method is provided for forming the system. For purposes of this description, “optical device” means a device with oculars or a camera and an objective lens through which a medical professional views a region of interest of a patient, for diagnostic or therapeutic purposes. In one embodiment, the optical device is an operating microscope (e.g. ophthalmic operating microscope).

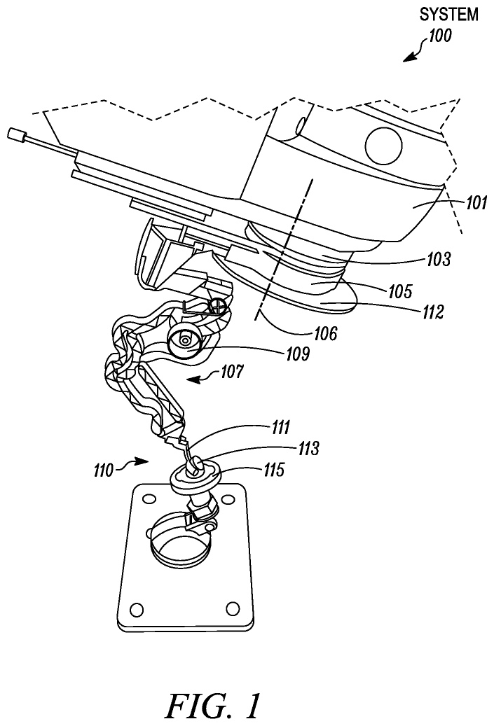

FIG. 1 is an image that illustrates an example of a system 100 providing a hands-free lens for use during optical surgery, according to an embodiment. In an embodiment, the system 100 includes a microscope 101 with an objective lens 103 that defines an objective optical axis 106. In an embodiment, the system 100 also includes an adapter plate 105 that is and a sterility disc 112 that covers the adapter plate 105 (e.g. to provide a sterile barrier for the microscope 101 and the adapter plate 105). In other embodiments, the system 100 excludes the microscope 101 and the adapter plate 105.

In an embodiment, the system 100 includes an optical attachment to attach a lens 113 to a microscope 101. For purposes of this description, “optical attachment” means one or more components that are used to independently or collectively position the lens 113 at a desired position, relative to the microscope 101. In one embodiment, the optical attachment includes a wide angle viewing attachment 107 (e.g. single use disposable). In an example embodiment, the wide angle viewing attachment 107 is the BIOM READY. In an example embodiment, a first end of the viewing attachment 107 is secured to the adapter plate 105 and a second end opposite to the first end is secured to a lens holder 111 that positions the lens 113 (e.g. on an eye 115). In some embodiments, the optical attachment also includes the lens holder 111. In one embodiment, the viewing attachment 107 has a knob 109 that can be adjusted (e.g. rotated) to vary a separation between the second end (e.g. the lens holder 111 and lens 113) and the microscope objective lens 103 (e.g. along the optical axis 106). In one embodiment, the system 100 includes the lens 113 (e.g. gonioscopy lens) and/or the optical attachment (e.g. the lens holder 111 and the viewing attachment 107) that is configured to move the lens 113 and the lens holder 111 relative to the microscope 101. In some embodiments, the lens 113 is a non-prismatic lens, a plano-concave lens, a mirrored lens, a double mirrored lens, a bioconcave lens or a combination thereof.

In one embodiment, the lens holder 111 and the lens 113 define an apparatus 110 that is used to position or couple the lens 113 to the microscope 101 such that the lens 113 has one or more degrees of freedom to accommodate relative movement between the eye 115 and the microscope 101 (e.g. translational degree of freedom to accommodate relative translational (axial) movement between the eye 115 and the microscope 101 and/or one or more rotational degrees of freedom to accommodate relative translational (lateral) movements between the eye 115 and the microscope 101). In the illustrated embodiment of FIG. 1, the lens 113 is a gonioscopy lens and sits on a model eye 115. However, in other embodiments where an actual patient is present, the lens holder 111 would similarly position the lens 113 on the eye of a patient (e.g. to gently rest on the cornea).

In an embodiment, the viewing attachment 107 (e.g. BIOM READY) is formed in such a way that the lens holder 111 and gonioscopy lens 113 are movable, essentially without resistance, in a direction towards the microscope objective (e.g. along the optical axis 106). In an example embodiment, where the viewing attachment 107 is the BIOM READY, the mechanics of the viewing attachment 107 is disclosed in U.S. Pat. No. 9,155,593, which is incorporated by reference herein. The inventors of the present invention recognized that this feature protects the eye from injury caused by the lens 113 during movement of the patient or movement of the microscope 101. FIG. 12 illustrates an example of a different view of the system 100 of FIG. 1, according to an embodiment.

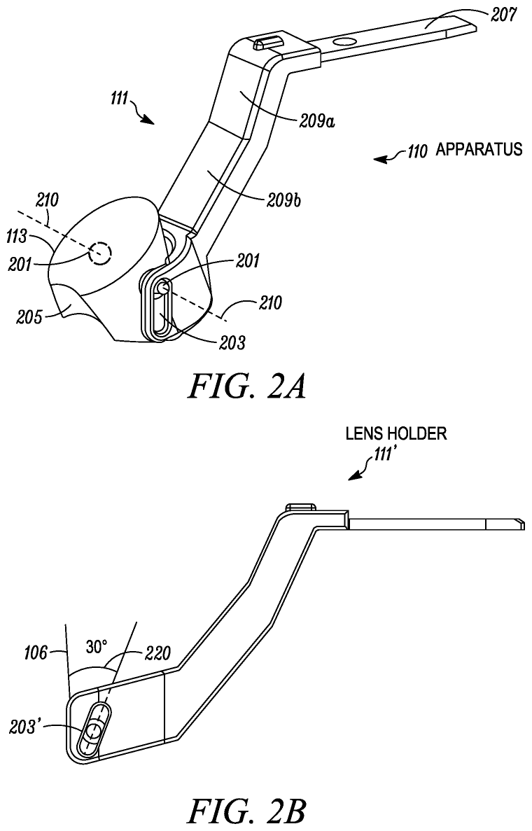

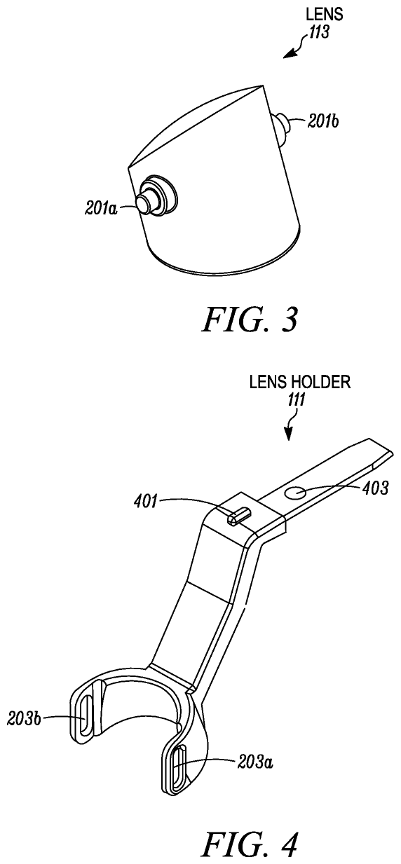

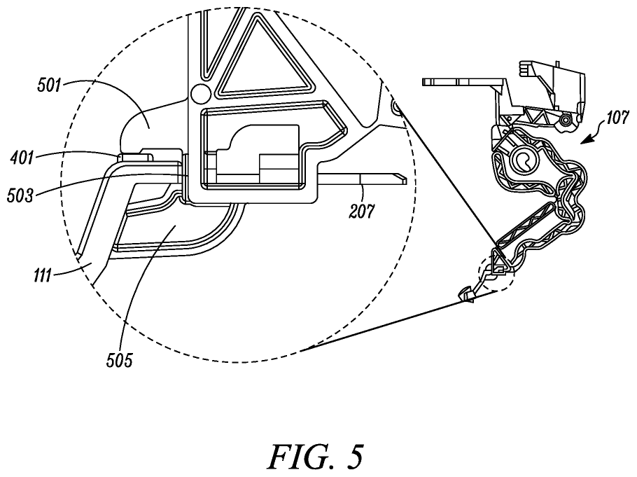

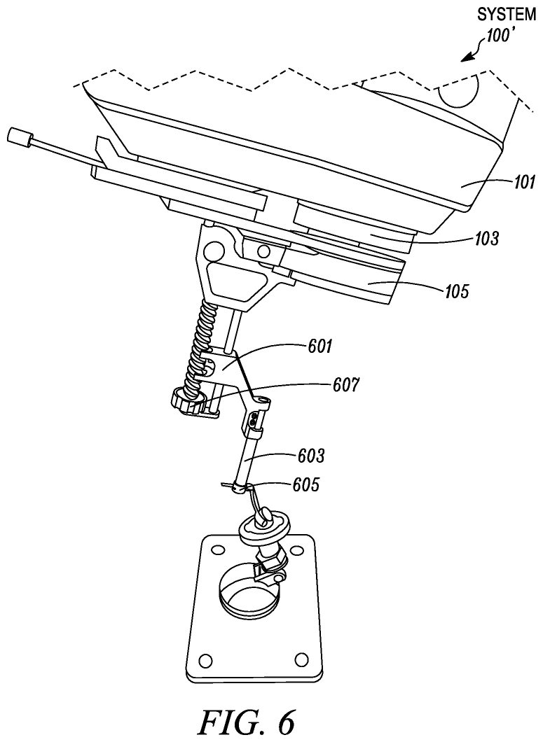

FIG. 2A is an image that illustrates an example of a side perspective view of the lens 113 and lens holder 111 of the system 100 of FIG. 1, according to an embodiment. FIG. 3 is an image that illustrates an example of a side perspective view of the lens 113 of FIG. 2A, according to an embodiment. FIG. 4 is an image that illustrates an example of a top perspective view of the lens holder 111 of FIG. 2A, according to an embodiment. In an embodiment, the lens 113 features a translation degree of freedom such that the lens 113 is configured to translate along a first direction (e.g. along the objective optical axis 106 and/or a direction with a component along the objective optical axis 106) relative to the microscope 101 and the optical attachment 107, 110. In an example embodiment, features of the lens 113 allow the lens 113 to safely be positioned on the cornea of the eye 115 (e.g. filleted edges and a biocompatible material minimize the risk of corneal injury or irritation). The inventors of the present invention recognized that this translational degree or freedom allows axial positioning of the lens 113 (e.g. along the objective optical axis 106) independent from the microscope 101 focus position.

In an embodiment, the apparatus 110 includes the lens holder 111 that defines a slot 203 configured to receive a portion of the lens 113 such that the lens is configured to translate within the slot 203 along the first direction. In one embodiment, FIG. 2A depicts that the slot 203 is oriented along the first direction (e.g. along the objective optical axis 106 and/or along a direction that is orthogonal to an axis defined by the first portion 207 of the lens holder 111). In another embodiment, FIG. 2B depicts a lens holder 211′ that is similar to the lens holder 211 of FIG. 2A, with the exception of the features discussed herein. In one example embodiment, the lens holder 211′ features a slot 203′ that is oriented at an angle 220 (e.g. at about 30 degrees or in a range from about 20 degrees to about 40 degrees and/or in a range from about 0 degrees to about 40 degrees) relative to the first direction (e.g. relative to the objective optical axis 106 and/or relative to a direction that is orthogonal to an axis defined by the first portion 207 of the lens holder 211).

In one example embodiment, the lens 113 includes a pair of posts 201a, 201b on opposite sides of the lens 113. In this example embodiment, the lens holder 111 defines a pair of slots 203a, 203b configured to receive the respective pair of posts 201a, 201b such that the pair of posts are configured to translate within the pair of slots 203a, 203b along the first direction. In other embodiments, the posts are provided on the lens holder 111 and the slots are provided on the lens 113. Although FIGS. 2-4 depict one structural arrangement (e.g. posts 201 of the lens 113 slidably received within slots 203, 203′ of the lens holder 111, 111′), the embodiments of the invention include any structural arrangement that facilitates the translational degree of freedom between the lens 113 and the lens holder 111.

In an example embodiment, a diameter of the slots 203 is slightly larger than the diameter of the posts 201, to advantageously permit the lens 113 to independently move in the first direction (e.g. along the objective optical axis 106). Additionally, the gonioscopy lens 113 features a cutout 205 which allows the surgeon surgical access to the eye.

In an embodiment, FIG. 3 depicts an embodiment of the lens 113 (e.g. prismatic gonioscopy lens) when it is not attached to the lens holder 111. In one example embodiment, the lens 113 is injection molded optically clear plastic such as Polymethyl methacrylate (PMMA), Polystyrene (PS), or Polycarbonate (PC). In another example embodiment, the lens 113 is machined glass or quartz/silica lens.

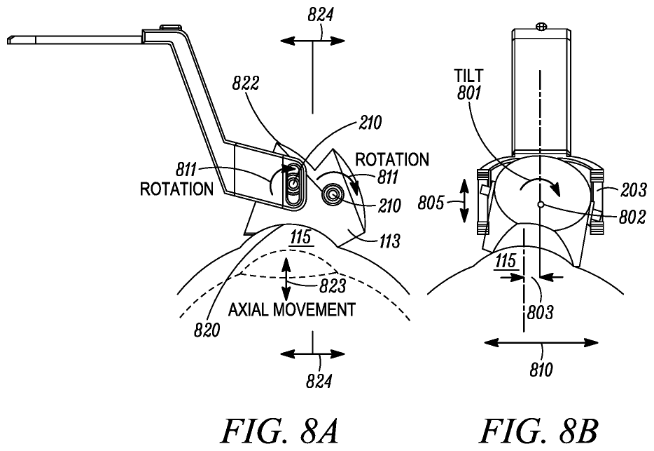

In one embodiment, FIG. 4 depicts the lens holder 111 when it is not retaining the gonioscopy lens 113. In another embodiment, the lens holder 111 includes retaining features 401, 403 which aid in attaching the lens holder 111 to the viewing attachment 107 (e.g. BIOM READY) or the viewing attachment 607 of FIG. 6 (e.g. OCULUS BIOM). In an example embodiment, the lens holder 111 is made from one or more of a glass-filled Polycarbonate (PC), Polypropylene (PP), or Acrylonitrile butadiene styrene (ABS). In another example embodiment, the lens holder 111 is made from a gamma-stable material. In yet another example embodiment, the lens holder 111 is made of a material with a flexural modulus (e.g. in a range from about 1 GPa to about 2 GPa).

In one embodiment, one or more characteristics of the lens 113 enable the lens 113 to be retained within the beam path (e.g. objective optical axis 106) of the microscope 101 by the lens holder 111. In an example embodiment, while retaining the lens 113, one or more characteristics of the lens holder 111 allow the lens 113 to pivot back-to-front, pivot side-to-side and/or move in a translational direction (e.g. vertically along the objective optical axis 106 for optimal positioning). In an example embodiment, the lens holder 111 also possesses features that allow it to interface and be retained by hardware typically used for a non-contact, wide-angle viewing lens for vitreoretinal procedures (e.g. the viewing attachment 107). In an example embodiment, one or more characteristics of the lens holder 111 also ensure alignment of the imaging lens 113 in the optical axis 106 and for positioning at the proper focal distance. In an example embodiment, a height of the lens holder 111 (e.g. defined as a dimension of the lens holder 111 along an axis that is orthogonal to the first portion 207) is about 22 millimeters (mm) or in a range from about 15 mm to about 30 mm. In another example embodiment, a length of the lens holder 111 (e.g. defined as a dimension of the lens holder 111 along an axis that is parallel to the first portion 207) is about 35 mm or in a range from about 30 mm to about 40 mm. In another example embodiment, the lens holder 111 includes angled portions 209a, 209b that are angled relative to the first portion 207 (FIG. 2A). In one example embodiment, the first angled portion 209a is oriented at a greater angle (relative to the first portion 207) then the second angled portion 209b. In an example embodiment, the first angled portion 209a is oriented at about 70 degrees or in a range from about 60 degrees to about 80 degrees relative to the first portion 207. In another example embodiment, the second angled portion 209b is oriented at about 55 degrees or in a range from about 40 degrees to about 70 degrees relative to the first portion 207. In still other embodiments, although two angled portions 209a, 209b are depicted in FIG. 2A in other embodiments, one angled portion (with the same orientation relative to the first portion 207) or more than two angled portions (e.g. with different respective orientations relative to the first portion 207) are provided in the lens holder 111. In still other embodiments, any geometry or design that connects the lower portion (e.g. horseshoe portion in FIG. 2A) to the upper first portion 207 (or retaining feature) can be utilized in the lens holder 111 design, provided that the connection between the lower portion and the upper first portion 207 is configured to position the lens 113 at the desired location during the surgical procedure when the lens holder 111 is secured to the attachment.

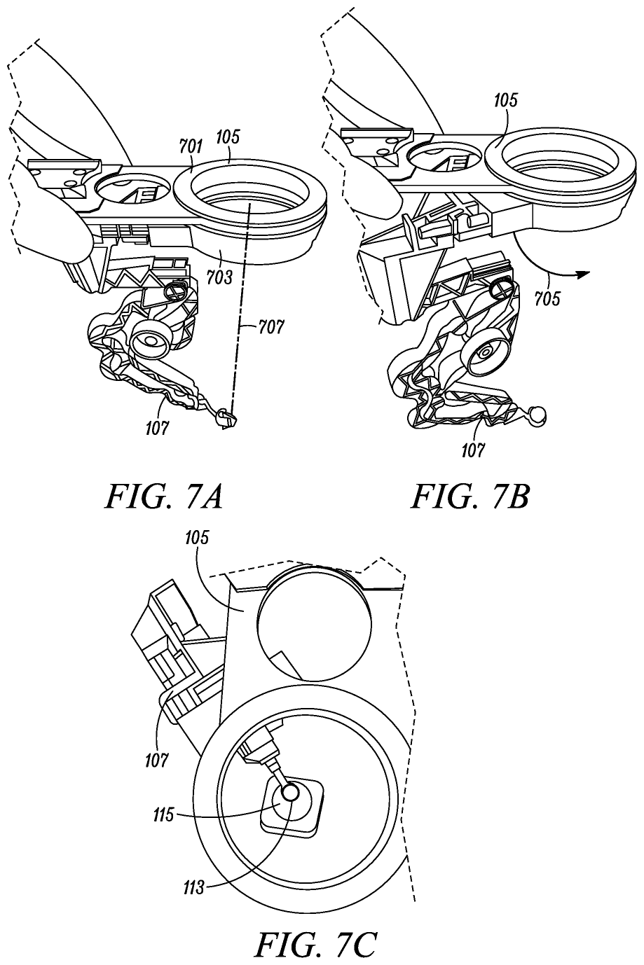

FIG. 5 is an image that illustrates an example of a side view of an attachment of the lens holder to the viewing attachment 107 of FIG. 1, according to an embodiment. In an embodiment, FIG. 5 depicts the interface between the lens holder 111 and the viewing attachment 107 (e.g. BIOM READY). In one embodiment, the lens holder 111 is inserted into a slot 503 of the viewing attachment 107. Upon full insertion, a feature of the viewing attachment 107 interferes with the feature 401 of the lens holder 111 (e.g. feature 501 interferes with feature 401 of the lens holder 111). This advantageously creates a frictional fit between the lens holder 111 and the viewing attachment 107 and aids to retain and stabilize the lens holder 111 and lens 113 to the viewing attachment 107. In another example embodiment, upon full insertion of the lens holder 111 into the slot 503, another feature 505 of the viewing attachment 107 interferes and sits flush with the corresponding face on the lens holder 111. In an example embodiment, this feature 505 also aids the user by providing a hard stop upon full insertion. In a yet further example embodiment, this feature 505 provides aid in the alignment and stable positioning of the gonioscopy lens 113 (e.g. relative to the viewing attachment 107 and/or the microscope 101).

FIG. 6 is an image that illustrates an example of a system 101′ providing a hands-free lens for use during optical surgery, according to an embodiment. In an embodiment, the system 100′ is similar to the system 100, with the exceptions of the features discussed herein. In an embodiment, unlike the viewing attachment 107 of the system 100 (e.g. disposable BIOM READY), the viewing attachment 601 of the system 100′ is a different viewing attachment (e.g. OCULUS BIOM). In the system 100′, the viewing attachment 601 is used to attach the apparatus 110 to the ophthalmic operating microscope 101 (e.g. with the adapter plate 105). As with the viewing attachment 107 of the system 100, the viewing attachment 601 of the system 100′ adjustably extends in an axial direction (e.g. along the objective optical axis 106) below the microscope objective lens 103. In an example embodiment, the viewing attachment 601 adjustably extends in the axial direction by rotating a knob 607.

In another example embodiment, the lens holder 111 is retained by the viewing attachment 601 when inserted into a slot 605 at the base of the viewing attachment 601. In this embodiment, when the lens holder 111 is fully inserted into the slot 605, a ball detent housed in a telescoping rod 603 of the viewing attachment 601 falls into place in the feature 403 along a first portion 207 of the lens holder 111. In an example embodiment, the feature 403 is a shallow divot that matches the geometry of the ball detent housed within the telescoping rod 603 of the viewing attachment 601. As with the viewing attachment 107 of the system 100, the telescoping rod 603 of the viewing attachment 601 allows the lens holder 111 and gonioscopy lens 113 to be movable, essentially without resistance, in a direction towards the microscope objective 103. In an example embodiment, the mechanics of the viewing attachment 601 are described in U.S. Pat. No. 7,092,152, which is incorporated by reference herein.

In an example embodiment, the viewing attachment 601 (as with the viewing attachment 107) is configured to move the lens 113 into contact with an eye 115 of a patient. In an example embodiment, the viewing attachment 601 includes an interface (e.g. knob 607) for manual adjustment of the position of the lens 113 relative to the microscope 101 (e.g. along the objective optical axis 106).

In yet another example embodiment, the lens 113 is configured to translate relative to the lens holder 111 in the first direction (e.g. along the objective optical axis 106) by a first extent and the lens 113 is configured to translate relative to the viewing attachment 601 by a second extent that is greater than the first extent. In an example embodiment, the first extent is about 3 mm or in a range from about 2 mm to about 4 mm. In another example embodiment, the second extent is about 35 mm or in a range from about 25 mm to about 45 mm.

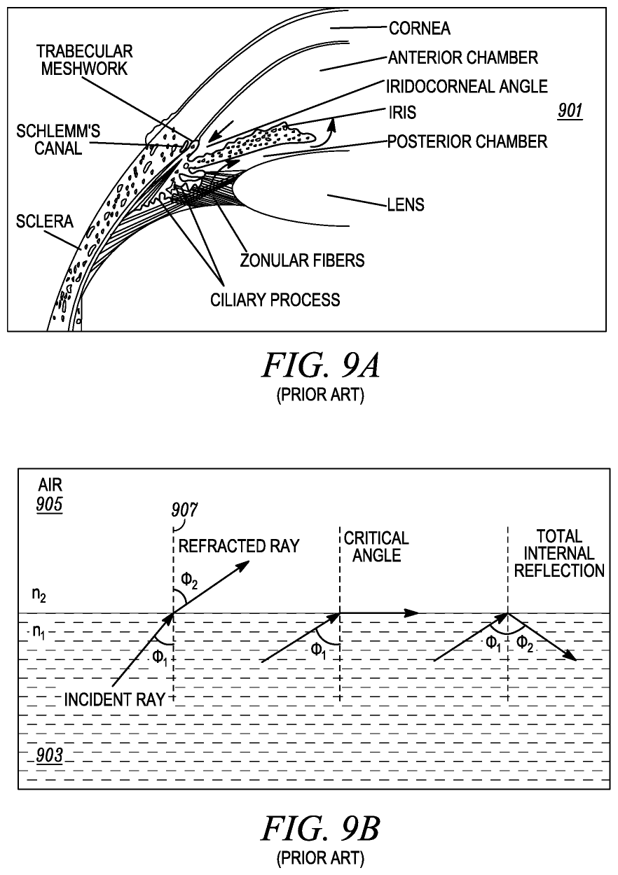

FIGS. 7A through 7C are images that illustrate an example of the viewing attachment 107 of FIG. 1 being rotated about an optical axis 106 of the microscope 101, according to an embodiment. Although the microscope 101 is not depicted in FIGS. 7A through 7C, the adapter plate 105 is depicted with an optical axis 707 that is typically aligned with the optical axis 106 of the microscope 101 when the adapter plate 105 is attached to the microscope 101. Although FIG. 7B depicts that the viewing attachment 107 is rotated in a counterclockwise direction 705, in other embodiments the viewing attachment 107 can also be rotated in a clockwise direction (e.g. opposite to the direction 705).

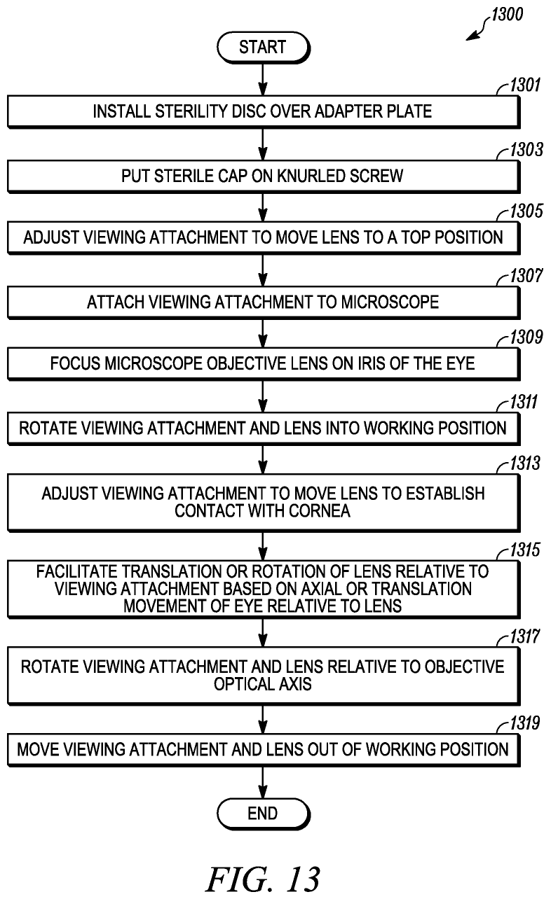

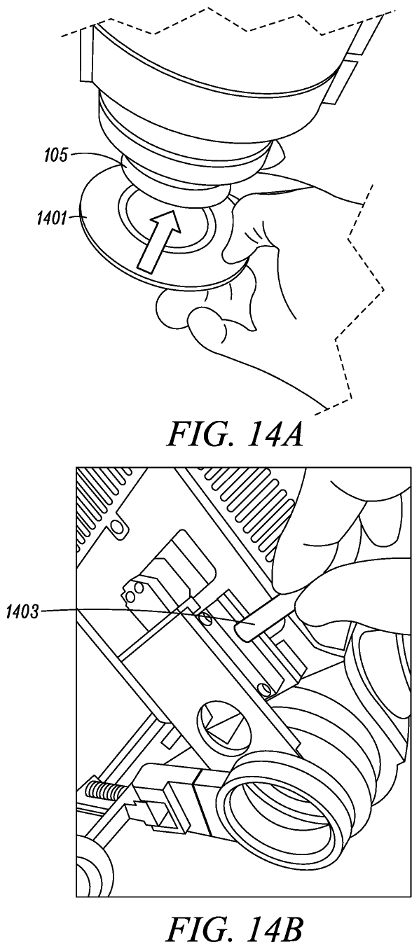

In an embodiment, the rotation of the viewing attachment 107 about the microscope objective axis 106 is provided, enabling the surgeon more field of view for those procedures where surgery is required at different circumferential regions of the eye. In an embodiment, FIGS. 7A through 7C show the adapter plate 105 in two different positions. In this embodiment, the adapter plate 105 is formed in two parts, an upper portion 701 and a lower portion 703 that is configured to pivot (e.g. about the axis 707 that is aligned with the microscope optical axis 106). In an example embodiment, the upper portion 701 is attached to the microscope 101. The upper portion 701 is subsequently locked into place while the lower portion 703 can rotate (e.g. over 360 degrees) about the optical axis 707. In the example embodiment, a method of using of the viewing attachment 107 involves using the rotating ability of the adapter 105 (e.g. up to about ±30 degrees in each direction to be able to rotate a gonioscopy lens 113 on the eye 115). This advantageously extends the viewing sector of the iridocorneal angle 901 (FIG. 9A). In FIGS. 7B and 7C, the lower portion 703 of the adapter 105 has been rotated a specific angle (e.g. 30 degrees) counterclockwise, and consequently the gonioscopy lens 113 has been rotated by this specific angle counterclockwise as well.

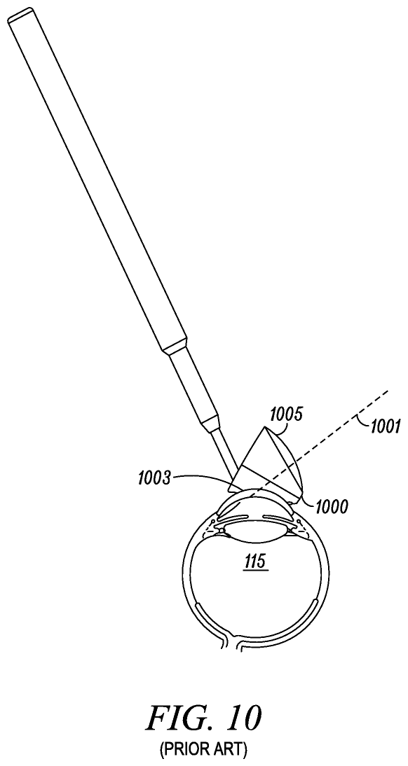

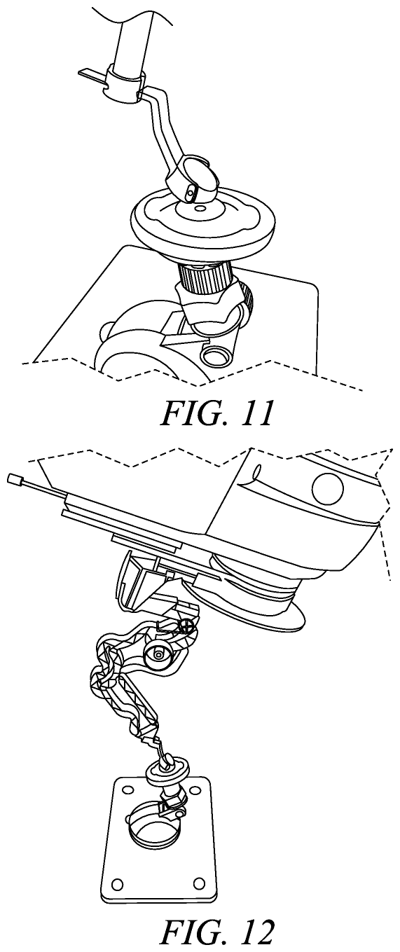

FIG. 8A is an image that illustrates an example of the lens of FIG. 1 configured to rotate about a first rotational axis 210 relative to the microscope 101, according to an embodiment. In an example embodiment, FIG. 8A is a side view of the lens 113 on the eye 115 and the rotation 811 about the first rotational axis 210 is a back/front pivot of the lens 113 (e.g. relative to the lens holder 111). FIG. 8B is an image that illustrates a cross-sectional view of FIG. 8A, showing the lens 113 configured to rotate about a second rotational axis 802 (e.g. extending out of the plane of the figure, about orthogonal to the plane of the figure, etc.) relative to the microscope 101, according to an embodiment. Unlike the rotation 811 about the first rotational axis 210 in FIG. 8A, the rotation 801 about the second rotational axis 802 in FIG. 8B is a side-to-side tilt (e.g. of the lens 113 within the lens holder 111).

In an embodiment, the first rotational axis 210 is angled relative to the first direction (e.g. translation direction of the lens 113 relative to the lens holder 111, such as along the slot 203 direction and the objective optical axis 106). In an example embodiment, the first rotational axis 210 is about orthogonal (e.g. about 90 degrees or in a range from about 70 degrees to about 110 degrees) to the first direction (e.g. objective optical axis 106). In an example embodiment, the first rotational axis 210 is defined by the posts 201a, 201b of the lens 113 (e.g. the axis 210 extends through the posts 201a, 201b). In another example embodiment, the second rotational axis 802 is angled (e.g. about orthogonal, such as about 90 degrees or in a range from about 70 degrees to about 110 degrees) relative to the first rotational axis 210 and/or the first direction (e.g. objective optical axis 106). In another example embodiment, the second rotational axis 802 is about orthogonal (e.g. about 90 degrees or in a range from about 70 degrees to about 110 degrees) relative to both the first rotational axis 210 and the first direction (e.g. objective optical axis 106).

In one embodiment, as shown in FIG. 8A the lens 113 includes a first surface 820 (e.g. bottom surface) that contacts the eye 115 (e.g. cornea). In an example embodiment, the first surface 820 has a curvature (e.g. concave surface) that is based on a curvature of the cornea so that the first surface 820 remains in contact with and concentric with the cornea (e.g. during the rotation about the axis 210). In an example embodiment, the curvature of the first surface 820 is about equal (e.g. within ±20%) of the curvature of the cornea. In an embodiment, the lens 113 includes a second surface 822 (e.g. top surface). In some embodiments, the lens 113 is a non-prismatic lens and/or a plano-concave lens (e.g. no angle between the axes of the first surface 820 and the second surface 822).

As shown in FIG. 8A, the lens 113 is positioned on the eye 115. In an example embodiment, the ophthalmic operating microscope 101 is tilted by a certain angle (e.g. about 30 degrees or in a range from about 20 degrees to about 50 degrees) from the vertical direction. In an example embodiment, this tilting of the microscope 101 is performed for surgeons use when performing a procedure involving the iridocorneal angle 901 (FIG. 9A). Thus, for other procedures (e.g. surgeries that do not involve the iridocorneal angle and/or viewing the eye for purposes other than surgery, such as diagnosis) the microscope 101 need not be tilted at this angle. In an embodiment, the first surface 820 is configured to remain in contact with the eye 115 so that light from within the eye 115 passes from the cornea into the lens 113 with minimal refraction (e.g. the difference between the index of refraction of the eye 115 and lens 113 is minimal at the interface between the eye 115 and lens 113). In an example embodiment, to prevent undesired refraction at the interface of the eye 115 and lens 113, solution is applied between the eye 115 and the lens 113 to reduce the instance of air gaps between the eye 115 and lens 113 (e.g. which would induce unwanted refraction at the eye/air and/or air/lens boundaries). In another example embodiment, the second surface 822 is angled such that incident light from within the lens 113 on the second surface 822 has a minimal incident angle relative to the normal to the second surface 822. The inventors of the present invention recognized that this minimalization of the incident angle on the second surface 822 reduces the likelihood that the incident light on the second surface 822 will undergo TIR and be reflected back into the lens 113 and instead will be transmitted through the second surface 822 and along the objective optical axis 106.

In one embodiment, the lens 113 is configured to be in contact and concentric with an eye 115 of a subject, such that the translational degree of freedom (e.g. along the first direction, such as along the objective optical axis 106 direction) and the first rotational degree of freedom (e.g. about the first rotation axis 210) is to accommodate movement of the eye in the first direction such that the lens 113 remains in contact and concentric with the eye 115 during this movement in the first direction. As shown in FIG. 8A, rotation of the lens 113 about the first rotational axis 210 is provided to maintain contact and concentricity in the case of axial displacement 823 in combination with lateral translational displacement 824.

In another embodiment, the lens 113 is configured to be in contact and concentric with the eye 115 of the subject such that the second rotational degree of freedom (e.g. about the second rotational axis 802) is configured to accommodate lateral movement of the eye 115 in a lateral direction orthogonal to the first direction such that the lens 113 remains in contact and concentric with the eye 115 during this movement in the lateral direction. In an example embodiment, FIG. 8B depicts a lateral displacement 803 which is accommodated by the rotation about the second rotational axis 802. In an example embodiment, the lateral displacement 803 is about ±4 mm or in a range from about ±1 mm to about ±6 mm. In yet another example embodiment, the axial displacement 823 is about ±4 mm or in a range from about ±1 mm to about ±6 mm.

In an embodiment, the rotation of the lens 113 about the second rotational axis 802 is based on the lens 113 pivoting about the second rotational axis 802 within the lens holder 111 (FIG. 8B). In an example embodiment, as shown in FIG. 8B, as the lens 113 pivots about the second rotational axis 802, the pair of posts 201a, 201b move in opposite directions within the respective pair of slots 203a, 203b. In an example embodiment, in order to accommodate this side-to-side tilt of the lens 113 within the lens holder 111, the inner width 810 (e.g. separation of the left and right inside surfaces of the lens holder 111) is greater than the width of the lens 113. In an example embodiment, to accommodate the side-to-side tilt 801, the inner width 810 of the lens holder 111 is about 20% greater than the diameter of the lens 113. In an example embodiment, the inner dimension of the lens holder 111 is about 0.3 mm wider (e.g. or in a range from about 0.1 mm to about 0.5 mm) wider than a width of a contacting feature at a base of the posts 201a, 201b. In another example embodiment, the side-to-side tilt angle of the lens 113 within the lens holder 111 is about ±15 degrees (side to side) or in a range from about ±10 degrees to about ±20 degrees.

In one embodiment, the first surface 820 (e.g. bottom surface contacting the eye 115) is a concave surface with a curvature that is based on a curvature of the eye such that the first surface is configured to be in contact and concentric with the eye. In another embodiment, the bottom/first surface 820 of the lens 113 is concave with a radius of curvature matching the radius of curvature of the cornea (e.g. about 8 mm or in a range from about 7 mm to about 9 mm) such that the lens 113 (e.g. made of a material with a similar index of refraction to the human cornea) minimizes the refractive power of the cornea. In another example embodiment, the second/top surface 822 of the lens 113 can have various designs each used to visualize a different region or anatomical feature within the eye and/or to control the magnification of the image. In one example embodiment, a second/top surface 822 is convex. In yet another example embodiment, the second/top surface 822 is angled by a certain angle (e.g. about 40 degrees or in a range from about 30 degrees to about 50 degrees). In other embodiments the lens 113 is a prismatic lens for gonioscopy. In still other embodiments, the lens 113 is a plano-concave lens, a bi-concave lens, and/or a convex-concave lens with spherical or aspheric surfaces. In some embodiments the lens 113 has an anti-reflective coating. In still other embodiments, the lens 113 is made of a gamma-stable material.

In an embodiment, a safety feature is provided by the apparatus 110, i.e. to allow intended (focusing) or unintended microscope movement without exerting a force onto the eye which could lead to injury. In an embodiment, the motion range of this safety feature should exceed the focus range necessary to view the eye structures to be examined, as well as exceed most expected unintended microscope movements. In an embodiment, this safety feature is achieved by a provision to allow tilt, rotation, and axial movement of the lens 113 to compensate for minor patient and eye movement, and allow lateral misalignment of the eye relative to the optical axis 106 of the microscope, to ensure continuous contact of the lens-cornea interface. The inventors of the present invention realized that this feature provides a constant, minimal contact force, in order to prevent compression of the anterior chamber during the procedure.

In yet another embodiment, a length 805 (FIG. 8B) of the slots 203 is adjusted to control a range of translational displacement of the lens 113 relative to the lens holder 111 a distance controlled by the length of the slots 203. In yet another embodiment, the diameter of the slots 203 are sized to be larger than the diameter of the posts 201, to accommodate the lens 113 independently pivoting about the first rotational axis 210 defined by the posts 201, and to independently tilt about the second rotational axis 802 (e.g. perpendicular to the first rotational axis 210 defined by the posts 201). In an example embodiment, the first rotational axis 210 is positioned above and approximately in line with a center of gravity of the lens 113 in order for the lens 113 to remain in the same rotational orientation when not in contact with the eye 115.

In an embodiment, the second surface 822 is angled at about 50 degrees (or in a range from about 40 degrees to about 60 degrees) relative to the first surface 820 to accommodate a wide range of microscope angles and eye anatomies in the visualization of the iridocorneal angle 901 (FIG. 9A).

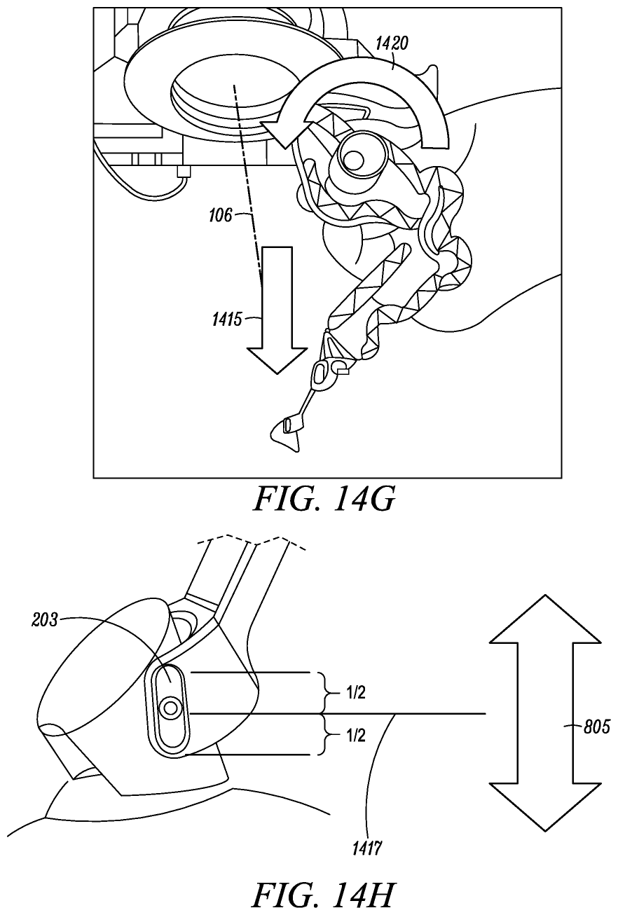

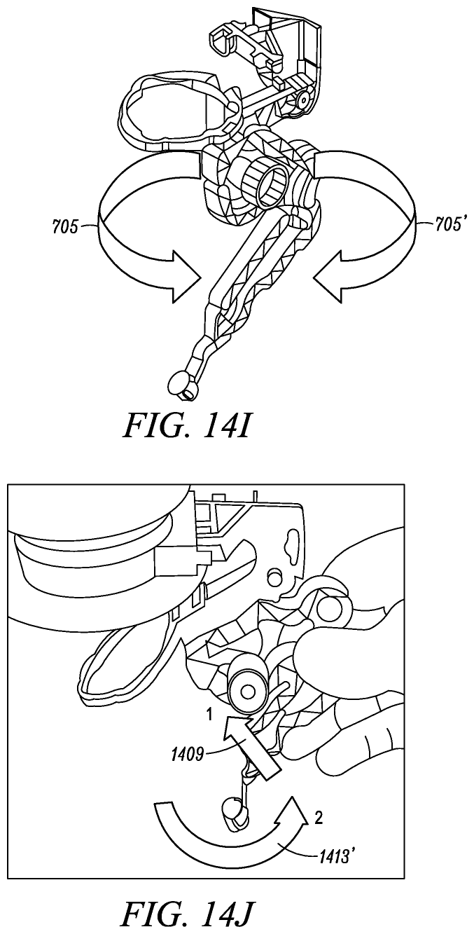

FIG. 13 is a flowchart that illustrates an example of a method 1300 for providing a hands-free lens for use during optical surgery, according to an embodiment. Although steps are depicted in FIG. 13 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways. FIGS. 14A through 14J are images that illustrate an example of one or more of the steps of the method 1300 being performed.

In an embodiment, in step 1301 a sterile barrier is positioned between the microscope 101 and the optical attachment (e.g. viewing attachment and/or lens holder). In one embodiment, in step 1301 a sterility disc 1401 (FIG. 14A) is positioned over the adapter plate 105 of the system 100. In an example embodiment, the sterility disc 1401 provides a sanitary barrier between the optical attachment (e.g. single use disposable) and the microscope 101 and/or the adapter plate 105 (e.g. non-disposable). In an example embodiment, in step 1301 the system 100 (excluding the microscope 101 and adapter plate 105) is removed from sterile packaging and includes the sterility disc 1401.

In an embodiment, in step 1303 a sterile cap 1403 is placed on a knurled screw (FIG. 14B). In one embodiment, in step 1303 the adapter plate 105 is secured to the microscope 101 by tightening the knurled screw (not labeled). In order to maintain sterility, a cap 1403 is placed over the knurled screw prior to adjusting.

In an embodiment, in step 1305 the lens 113 and lens holder 111 are secured to the viewing attachment. In one embodiment, in step 1305 the lens holder 111 is secured to the viewing attachment 107, 601 using various features 401, 403 as discussed in the embodiment of FIG. 5. In one embodiment, in step 1305 after the lens 113 and lens holder 111 are secured to the viewing attachment 107, 601, the viewing attachment 107, 601 is adjusted so that the lens 113 is at a top position (e.g. maximum position of a range of movement of the viewing attachment in an upward direction). In an example embodiment, in step 1305 the knob 109 of the viewing attachment 107 is rotated in a first direction 1407 (FIG. 14C) so to move the lens 113 and lens holder 111 in a first direction 1409. In this example embodiment, the knob 109 is rotated in the direction 1407 until the lens 113 is at a top position of the viewing attachment 107. In other embodiments, in step 1305 the viewing attachment 601 is used and the knob 607 is rotated until the lens 113 is at the top position.

In an embodiment, in step 1307 the viewing attachment is attached to the microscope 101. In one embodiment, in step 1307 a portion of the viewing attachment 107 (FIG. 14D) is moved in a direction 1411 such that the portion of the viewing attachment 107 is received in a slot of the adapter plate 105. In another embodiment, in step 1307 the viewing attachment 601 is secured to the adapter plate 105 using a similar technique as depicted in FIG. 14D for the viewing attachment 107. In an example embodiment, in step 1307 the portion of the viewing attachment 107 is moved into the slot of the adapter plate 105 until the viewing attachment 107 is securely engaged to the adapter plate 105.

In an embodiment, in step 1309 the objective lens 103 of the microscope 101 is focused to the proper distance. In one embodiment, in step 1309 the objective lens 103 is focused on the iris of the eye 115.

In an embodiment, in step 1311 the viewing attachment is moved to a working position (e.g. a position where the surgeon can view the eye for purposes of performing one or more surgical procedures and/or can diagnose one or more conditions of the eye). In one embodiment, in step 1311 the viewing attachment 107, 601 (e.g. and the lens 113 and lens holder 111) is rotated in a direction 1413 (FIG. 14F) to the working position. In an example embodiment, the working position is a position where the lens 113 is aligned with the objective optical axis 106 of the objective lens 103. As shown in FIG. 14F, in one embodiment, after moving the lens 113 and lens holder 111 in the direction 1409 to the top position (step 1305) the viewing attachment 107 is rotated in the direction 1413 until the lens 113 and lens holder 111 are in the working position.

In an embodiment, in step 1313 the viewing attachment is adjusted to move the lens 113 to establish contact with the eye 115 (e.g. cornea). In one embodiment, in step 1313 the viewing attachment 107 is adjusted by rotating the knob 109 in a second direction 1420 (FIG. 14G) that is opposite to the first direction 1407 of step 1305 (FIG. 14C). In an example embodiment, in step 1313 the viewing attachment is adjusted such that the lens 113 is slowly lowered in a downward direction 1415 (FIG. 14G) that is opposite from the upward direction 1409 in step 1305. In an example embodiment, in step 1313 the adjustment of the viewing attachment is stopped once the lens 113 has made full contact with the cornea. In an example embodiment, in step 1313 once full contact is established between the lens 113 and the cornea, the focus of the microscope objective lens 103 is adjusted to optimize a view of the eye 115 at the iridocorneal angle 901 with the lens 113 (e.g. to correct for change in the optical path length with the lens 113 in place). In an example embodiment, in step 1313 focusing down may result in a slight compression of the flexible lower arm and a temporary increase in the pressure of the eye. In an example embodiment, the method 1300 advantageously ensures that the pressure of the lens 113 on the eye 115 does not exceed a threshold force (e.g. about 1 Newton (N)).

In an embodiment, in step 1313 the viewing attachment is adjusted to move the lens 113 such that the posts 201a, 201b of the lens 113 are positioned within the slots 203 of the lens holder 111 to facilitate axial movement of the lens 113 relative to the lens holder 111 (and viewing attachment). In an example embodiment, in step 1313 the viewing attachment is adjusted until the posts 201a, 201b of the lens 113 are positioned at about a midpoint 1417 (FIG. 14H) of a range of the slot 203. The inventors of the present invention recognized that this advantageously maximizes a range of axial displacement 823 and side-to-side tilt 801 of the lens 113 relative to the lens holder 111 (e.g. in both the upward direction 1409 and downward direction 1415).

In an embodiment, in step 1315 relative movement between the lens 113 and the lens holder 111 and/or viewing attachment is facilitated, in one or more degrees of freedom while still ensuring that the lens 113 remains in contact with and/or concentric with the cornea. In one embodiment, in step 1315 relative translational movement between the lens 113 and the lens holder 111 (and/or viewing attachment) is facilitated based on translational movement of the posts 201 within the slot 203 of the lens holder 111. In still other embodiments, in step 1315 relative rotational movement between the lens 113 and the lens holder 111 and/or viewing attachment is facilitated about the first rotational axis 210 (e.g. to facilitate front/rear pivot of the lens 113). In still other embodiments, in step 1315 relative rotational movement between the lens 113 and the lens holder 111 and/or viewing attachment is facilitated about the second rotational axis 802 (e.g. to facilitate side-to-side tilt of the lens 113).

In an embodiment, in step 1317 the viewing attachment (and lens 113) is rotated relative to the microscope 101. In one embodiment, in step 1317 the viewing attachment 107 is rotated about the optical axis 106 of the microscope objective lens 103. In an example embodiment, the viewing attachment 107 is rotated in a counterclockwise direction 705 (FIG. 14I) or a clockwise direction 705′. In an embodiment, step 1317 is performed to achieve an expanded view of the interior chamber of the eye at an angle (e.g. iridocorneal angle 901). In an example embodiment, in step 1317 the viewing attachment is rotated within an angular range (e.g. about 30 degrees) in the directions 705, 705′. In another example embodiment, during the rotation of step 1317, the lens 113 is not separated from the cornea, and thus provides the surgeon with an expanded view of the eye 115 along the angle (e.g. an expanded view of the anterior chamber of the eye at the iridocorneal angle 901, where the anterior chamber can be viewed at the angle 901 from different orientations, etc.).

In an embodiment, in step 1319 the viewing attachment is moved out of the working position. In one embodiment, step 1319 is performed after the procedure (e.g. eye surgery). In an embodiment, in step 1319 the viewing attachment 107 (and lens 113) are moved out of the working position. In an example embodiment, step 1319 is a reverse of step 1311, where the viewing attachment 107 is adjusted to move the lens 113 in the upward direction 1409 and/or is rotated about a direction 1413′ (FIG. 14J) to move the viewing attachment 107 (and lens 113) out of the working position.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

What is claimed is:

1. An apparatus for attaching a lens to a microscope with an optical attachment, said apparatus comprising:
the lens with a translational degree of freedom such that the lens is configured to translate along a first direction relative to the microscope and the optical attachment.

2. The apparatus as recited in claim 1, wherein said microscope includes an objective lens that defines an objective optical axis, and wherein the first direction is along the objective optical axis.

3. The apparatus as recited in claim 1, wherein said microscope includes an objective lens that defines an objective optical axis, and wherein the first direction is along the optical axis of the eye.

4. The apparatus as recited in claim 1, wherein the optical attachment includes a lens holder and a positioning device to move the lens and the lens holder relative to the microscope;
wherein the apparatus further includes the lens holder that defines a slot configured to receive a portion of the lens such that the lens is configured to translate within the slot along the first direction.

5. The apparatus as recited in claim 4, wherein the portion of the lens is a pair of posts on opposite sides of the lens and wherein the slot defined by the lens holder is a pair of slots configured to receive the respective pair of posts such that the pair of posts are configured to translate within the pair of slots along the first direction.

6. The apparatus as recited in claim 1, wherein the lens includes a first rotational degree of freedom such that the lens is configured to rotate relative to the microscope and the optical attachment about a first rotational axis that is angled with respect to the first direction.

7. The apparatus as recited in claim 6, wherein the first rotational axis is orthogonal to the first direction.

8. The apparatus as recited in claim 6, wherein the lens includes a second rotational degree of freedom such that the lens is further configured to rotate relative to the microscope and the optical attachment about a second rotational axis that is angled with respect to the first direction and the first rotational axis.

9. The apparatus as recited in claim 8, wherein the first rotational axis is orthogonal to the first direction and the second rotational axis is orthogonal to the first rotational axis and the first direction.

10. The apparatus as recited in claim 8, wherein the lens is configured to be in contact and concentric with an eye of a subject, such that:
the translational degree of freedom is to accommodate movement of the eye in the first direction such that the lens remains in contact and concentric with the eye during said movement in the first direction; and
the first and second rotational degree of freedom is configured to accommodate lateral movement of the eye in a lateral direction orthogonal to the first direction such that the lens remains in contact and concentric with the eye during said movement in the lateral direction.

11. The apparatus as recited in claim 8, wherein the optical attachment includes a lens holder and a positioning device to move the lens and the lens holder relative to the microscope;
wherein the apparatus further includes the lens holder that defines a pair of slots configured to receive a respective pair of posts on opposite sides of the lens such that the lens is configured to translate within the pair of slots along the first direction; and
wherein the first rotational axis is defined by the pair of posts of the lens.

12. The apparatus as recited in claim 11, wherein the second rotational degree of freedom is based on the lens pivoting about the second rotational axis within the lens holder such that the pair of posts move in opposite directions within the pair of slots.

13. The apparatus as recited in claim 1, wherein the lens has a first surface configured to be in contact with an eye of a patient and a second surface opposite to the first surface.

14. The apparatus as recited in claim 13, wherein the first surface is a concave surface with a curvature that is based on a curvature of the eye such that the first surface is configured to be in contact and concentric with the eye.

15. The apparatus as recited in claim 13, wherein the first surface is in contact and concentric with the eye and wherein the second surface is configured such that incident light on the second surface from an interface of the first surface and the eye does not undergo total internal reflection.

16. The apparatus as recited in claim 15, wherein the lens is a non-prismatic lens.

17. A system comprising:
the lens of claim 1; and
the optical attachment of claim 1 comprising a lens holder and a positioning device configured to move the lens and the lens holder relative to the microscope.

18. The system of claim 17 wherein the positioning device is configured to move the lens in contact with an eye of a patient.

19. The system of claim 17, wherein the positioning device comprises an interface for adjustment of the position of the lens relative to the microscope.

20. The system of claim 17, wherein the microscope includes an objective lens that defines an objective optical axis and wherein the positioning device is configured to move the lens along the objective optical axis.

21. The system of claim 17 wherein the lens is configured to translate relative to the lens holder in the first direction by a first extent and wherein the lens is configured to translate relative to the positioning device in the first direction by a second extent greater than the first extent.

22. The system of claim 17, wherein the lens holder defines a slot configured to receive a portion of the lens such that the lens is configured to translate within the slot along the first direction.

23. A method for using an optical attachment to position a lens relative to a microscope, comprising:
securing the lens to a first end of the optical attachment;
securing a second end of the optical attachment to the microscope;
moving, with the optical attachment, the lens until the lens makes contact with an eye of a patient; and
translating the lens along a first direction relative to the microscope and the optical attachment, based on relative movement of the eye in the first direction such that the lens maintains contact with the eye.

24. The method according to claim 23, wherein the optical attachment includes a lens holder and a positioning device;
wherein the securing the lens includes securing the lens to the lens holder of the optical attachment and wherein the translating step comprises translating the lens relative to the lens holder.

25. The method according to claim 23, wherein the microscope includes an objective lens that defines an objective optical axis;
wherein the securing the second end comprises securing the positioning device to the microscope;
wherein the moving step comprises moving, with the positioning device, the lens along the objective optical axis until the lens makes contact and is concentric with the eye of the patient;
wherein the translating includes one of translating the lens relative to the lens holder in the first direction by a first extent or translating the lens and the lens holder relative to the positioning device by a second extent that is greater than the first extent.

26. The method according to claim 23, wherein the moving step comprises moving the lens until the lens makes contact with the eye and the lens is within a middle of a range of the translating in the first direction.

27. The method according to claim 25, further comprising pivoting the positioning device, the lens holder and the lens about the objective optical axis.
